(19) United States
(12) Patent Application Publication
(10) Pub. No.: US 2020/0121180 A1
(43) Pub. Date: Apr. 23, 2020

(54) DIAGNOSTIC EYEWEAR FOR EVALUATION OF AND ADJUSTMENT TO MONOVISION

(71) Applicant: Christopher Harris, Oakdale, PA (US)

(72) Inventor: Christopher Harris, Oakdale, PA (US)

(21) Appl. No.: 16/659,954

(22) Filed: Oct. 22, 2019

Related U.S. Application Data
(60) Provisional application No. 62/748,568, filed on Oct. 22, 2018.

(51) Int. Cl. A61B 3/04; A61B 3/09

(52) U.S. Cl. CPC A61B 3/04; A61B 3/09

(57) ABSTRACT

Diagnostic eyewear systems are disclosed that facilitate the evaluation of and adjustment to monovision as a solution for near vision loss experienced by corrected nearsighted and normal vision persons. The diagnostic eyewear include a magnifying lens and a non-magnifying lens, and a frame that allows the user to switch the locations of the magnifying and non-magnifying lenses from one eye to another. Multiple diagnostic eyewear may be provided as a set, with the magnifying lens of each eyewear having a different magnification power. The diagnostic eyewear may be used to determine magnification eye preference, magnification strength and distance eye preference in order to facilitate acclimation to monovision.

### CROSS-REFERENCE TO RELATED APPLICATION

The present application claims priority from U.S. Provisional Patent Application No. 62/748,568 filed on Oct. 22, 2018, which is incorporated herein by reference.

### FIELD OF THE INVENTION

The present invention relates to diagnostic eyewear systems that facilitate the evaluation of and adjustment to monovision as a solution for near vision loss experienced by corrected nearsighted and normal vision persons. The diagnostic eyewear may be used to determine magnification eye preference, magnification strength and distance eye preference in order to facilitate acclimation to monovision.

### BACKGROUND OF THE INVENTION

At the age of about 40 years old, many people develop a need for near vision correction due to a condition known as presbyopia. Persons requiring glasses or contact lenses for distance vision are commonly referred to as being nearsighted. When a nearsighted person develops presbyopia with age, one solution is to prescribe glasses with bifocal lenses in which the upper portion of each lens corrects for nearsightedness and the bottom portion of each lens provides correction for better near vision, e.g. for reading. Normal vision persons typically use reading glasses to overcome presbyopia and provide better near vision.

Other solutions include progressive bifocal lenses, multiple pairs of corrective lenses, such as distance vision contact lenses and reading glasses, two or more pairs of glasses, multifocal contact lenses and monovision.

Monovision allows a person to have two different corrections, one in each eye, to correct for different purposes. For example, a person could have correction for nearsightedness in one eye and for improved near vision in the other eye. Monovision correction can be obtained through contact lenses, glasses, or surgery.

A person desiring monovision must choose which eye is corrected for which purpose. For example, a person may have a preference for the right eye corrected for nearsightedness and the left eye corrected for near vision, or vice versa. A similar choice is also required for prescribing multifocal contact lenses.

When a person is tested for this preference at a doctor's office, the doctor has a short time to determine the eye preference. The doctor may choose the person's non-dominant eye for near vision correction, but some people prefer the dominant eye for near vision correction.

### SUMMARY OF THE INVENTION

Diagnostic eyewear systems are provided that facilitate the evaluation of and adjustment to monovision as a solution for near vision loss experienced by corrected nearsighted and normal vision persons. The diagnostic eyewear include a magnifying lens and a non-magnifying lens, and a frame that allows the user to switch the locations of the magnifying and non-magnifying lenses from one eye to another. A set of such eyewear is provided with the magnifying lens of each eyewear having a different magnification power. The diagnostic eyewear may be used to determine magnification eye preference, magnification strength and distance eye preference in order to facilitate acclimation to monovision.

An aspect of the present invention is to provide diagnostic eyewear for determining magnification eye preference for a wearer of the diagnostic eyewear to facilitate acclimation of the wearer to monovision, the diagnostic eyewear comprising a frame, a first non-magnifying portion of the frame, and a second magnifying lens supported by the frame, wherein the frame is structured and arranged to alternatively position the first non-magnifying lens in a left viewing position in front of a left eye of the wearer while the second magnifying lens is positioned in a right viewing position in front of a right eye of the wearer, and then to position the first non-magnifying lens in the right viewing position while the second magnifying lens is positioned in the left viewing position. The first non-magnifying portion of the frame may include a non-magnifying lens that is supported by the frame.

Another aspect of the present invention is to provide a method of determining magnification eye preference to facilitate acclimation to monovision using the diagnostic eyewear described above. The method comprises placing the diagnostic eyewear on the wearer in a first one of the alternative positions, and subsequently switching the position of the diagnostic eyewear to the other alternative position to thereby determine the magnification eye preference of the wearer.

A further aspect of the present invention is to provide a diagnostic eyewear kit comprising a plurality of diagnostic eyewear, wherein each of the diagnostic eyewear comprises a frame, a first non-magnifying portion of the frame, and a second magnifying lens supported by the frame, wherein the frame is structured and arranged to alternatively position the first non-magnifying lens in a left viewing position in front of a left eye of the wearer while the second magnifying lens is positioned in a right viewing position in front of a right eye of the wearer, and then to position the first non-magnifying lens in the right viewing position while the second magnifying lens is positioned in the left viewing position, and wherein the second magnifying lens of each of the diagnostic eyewear has a different magnification power than the other second magnifying lenses. The first non-magnifying portion of the frame may include a non-magnifying lens that is supported by the frame.

Another aspect of the present invention is to provide a method of determining desired magnification strength to facilitate acclimation to monovision using the diagnostic eyewear kit described above. The method comprises placing a first diagnostic eyewear on the wearer in which the second magnifying lens has a first magnification power, and placing a second diagnostic eyewear on the wearer in which the second magnifying lens has a second magnification power different from the first magnification power to thereby determine the desired magnification strength for the wearer.

These and other aspects of the present invention will be more apparent from the following description.

### BRIEF DESCRIPTION OF THE DRAWINGS

FIG. 1 is a partially schematic isometric view of diagnostic eyewear in accordance with an embodiment of the present invention.

FIG. 2 is a partially schematic isometric view of diagnostic eyewear in accordance with another embodiment of the present invention wherein the lenses are round-shaped.

FIG. 3 is a partially schematic isometric view of diagnostic eyewear in accordance with a further embodiment of the present invention.

FIG. 4 is a partially schematic top view of diagnostic eyewear in accordance with an embodiment of the present invention.

FIG. 5 is a partially schematic top view of diagnostic eyewear in accordance with another embodiment of the present invention.

FIG. 6 is a partially schematic front view of a set of diagnostic eyewear in accordance with an embodiment of the present invention wherein one lens of each eyewear of in the set is non-magnifying and the other lens is magnifying with a different strength.

FIG. 7 is a partially schematic isometric view of diagnostic eyewear in accordance with an embodiment of the present invention.

### DESCRIPTION

The present invention provides a system for evaluating monovision through simulation using diagnostic eyewear. The system is useful for contact lens wearers, eyeglass wearers and normal vision persons to determine for which eye, dominant or non-dominant, to provide near vision correction. The present invention is also useful for those undergoing surgery, such as Lasik or cataract surgery, to achieve monovision correction. The present invention may further be useful for multifocal contact lens wearers to determine which eye's correction is biased toward distance vision and which eye's correction is biased toward near vision. It may take time for a person to realize the near vision correction eye preference and the correction strength preference. If a person's preference for either the near vision correction eye or correction strength is incorrect, this could be costly to the person and the doctor, in terms of time and sample contact lenses. The present invention provides a system where a person can simulate and evaluate monovision and near vision correction strength and eye preference prior to finalizing a presbyopia solution.

The present invention further provides to a method wherein a wearer is provided diagnostic eyewear, and wears the glasses in a first position for an amount of time and then switches to a second position for an amount of time. The amounts of time may range from a minute or less to a day or more, e.g., from several minutes to several hours. The wearer may optionally switch between the first position and the second position until the wearer determines which eye is preferred for magnification.

The present invention further provides a system of diagnostic eyewear as described herein. A wearer may be provided with a kit or set two or more diagnostic eyewear, such as a set of three to five pairs of diagnostic eyeglasses, wherein the second lens of each diagnostic eyewear has a different magnification strength than the other diagnostic eyewear in the set. The diagnostic eyewear sets may be provided in multiple pupillary distances to allow better vision correction for varying patients.

The present invention further relates to a method wherein a wearer is provided a kit or set of diagnostic eyewear, and the wearer wears at least two of the diagnostic eyewear in the set for a predetermined period of time. After evaluating at least two of the diagnostic eyewear in the set, the wearer determines the magnification strength preference for the preferred near vision eye.

In accordance with embodiments of the present invention, diagnostic eyewear in the form of a pair of eyeglasses are switched from a first position to a second position, e.g., by rotating the eyeglasses 180° along an axis of rotation corresponding to the wearer's line of sight, or by rotating the temples of the eyeglasses 180° from backward extending to forward extending. For each diagnostic eyewear, a frame may be provided with a first lens that may be non-magnifying and non-corrective, e.g., a plano lens, and a second lens that is magnifying. Alternatively, the first lens, being non-corrective, may be omitted from the frame of the eyeglasses. The first non-magnifying lens may be corrective in certain embodiments. The frame may have a nosepiece or nose bridge and temples such that the frame is structured and arranged to be worn with each lens on either the left or right side of the wearer. In addition to being 180° rotationally symmetric about the line of sight axis, the frames may be 180° symmetric about a horizontal axis that is perpendicular to, and intersects, the line of sight axis. Thus, when viewed from the front, the top half of a frame and the bottom half of the frame may appear as identical mirror images of each other. The frames and lenses can be of any shape, such as oval, round, rectangular, or the like as long as the lenses can be worn on either the user's left or right eye. As shown in FIGS. 1, 2 and 3, and more fully described below, diagnostic eyewear 10, 110 and 210 may include various types of lens shapes.

FIG. 1 is a partially schematic isometric view illustrating diagnostic eyewear 10 in accordance with an embodiment of the present invention. The diagnostic eyewear 10 includes a frame 12 having a first temple piece 13, second temple piece 14, and nose bridge 15. The temple pieces 13 and 14 of the diagnostic eyewear 10 may be substantially straight, or may be curved, e.g., in a horizontal plane. The nose bridge 15 may be symmetrical around a horizontal axis that is perpendicular to the line of sight of the wearer. The frame 12 may also include a first lens frame 16 and a second lens frame 17 connected together by the nose bridge 15. In the embodiment shown in FIG. 1, the first and second lens frames 16 and 17 are ovular. The first and second temple pieces 13 and 14 may be connected by any suitable means to the first and second lens frames 16 and 17, respectively, e.g., by hinges.

In the embodiment shown in FIG. 1, The diagnostic eyewear 10 includes a first lens 21 which is non-magnifying, and a second lens 22 which is magnifying. The first and second lenses 21 and 22 may be held in place by the first and second lens frames 16 and 17. Alternatively, the first and second lens frames 16 and 17 may be fully or partially eliminated, e.g., the diagnostic eyewear 10 may be frameless, in which case the first and second lenses 21 and 22 are attached directly to the first and second temple pieces 13 and nose bridge 15. The diagnostic eyewear 10 may also be partially frameless, e.g., the first and second lens frames may extend only over portions of the edges of the first and second lenses 21 and 22. In certain embodiments, the first lens 21 may be eliminated from the first lens frame 16, i.e., the first lens frame may be open to provide a first non-magnifying portion.

As shown in FIG. 1, the frame 12 defines a line of sight axis S that extends forward through the center of the nose bridge 15 in a direction parallel with the wearer's line of vision. The frame 12 also defines a horizontal bisection line H that intersects the line of sight axis S and divides the frames 12 into upper and lower halves that are mirror images of each other in the embodiment shown in FIG. 1.

As shown in FIG. 1, the frame is 180° rotationally symmetric about the line of sight axis S of the eyewear. In addition, the frame is 180° rotationally symmetric about the horizontal bisection line H that intersects the line of sight axis. The nose bridge 15 may be structured and arranged so a person can wear the diagnostic eyewear 10 in the first position and the second position with uniformity and comfort. For example, where the second position is obtained by rotating the diagnostic eyewear 10, the nose bridge 15 may be positioned at the center of the lenses so that the frame 10 is symmetrical about the horizontal bisection line H. The frame 12 may also be structured to minimize vertex distance between the wearer's selected eye and the second magnifying lens 22 lens in order to minimize the magnification effect inherent in plus powered lenses.

The first lens 21 is typically non-magnifying and non-corrective, e.g., a plano lens. However, a person of skill in the art would recognize that a person may require diagnostic eyewear where the first lens 21 is corrective for distance vision. For example, a person who does not wear contact lenses but uses glasses for distance vision correction may desire monovision with contact lenses or surgery. Vision correction may be provided in the non-magnified first lens 21 of the diagnostic eyewear, such as for nearsightedness.

The second lens 22 is typically magnifying, and may be provided with any suitable magnification power. For example, the second magnifying lens 22 may have a magnification power of less than +0.25 to more than +10.00 and any strength therebetween. While the second lens may typically be non-corrective for distance, certain users may benefit from a corrective second lens, such as a person who wears eyeglasses for distance vision correction but may desire monovision with contact lenses or surgery.

As more fully described below, when the diagnostic eyewear 10 is provided in a set, the set has at least two of the diagnostic eyewear, wherein the second lens of each diagnostic eyewear has a different magnification from the second lens(es) of the other diagnostic eyewear. For example, the set may include two to six or more of diagnostic eyewear, and the second magnifying lens of each eyewear may have a magnification of less than +0.25 to more than +10.00, and any strength therebetween.

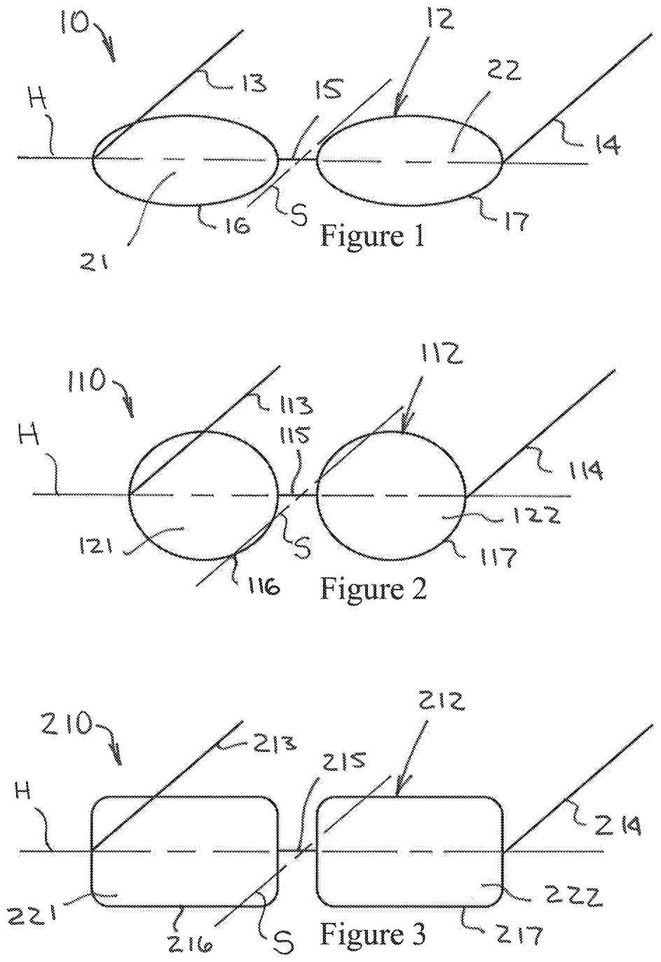

FIG. 2 is a partially schematic isometric view illustrating diagnostic eyewear 110 in accordance of another embodiment of the present invention. Other than having generally circular lens frames, the features of the diagnostic eyewear 110 shown in FIG. 2 are similar to the features of the diagnostic eyewear 10 shown in FIG. 1. The diagnostic eyewear 110 includes a frame 112 having a first temple piece 113, second temple piece 114, and nose bridge 115. The first and second temple pieces 113 and 114 may be connected to the first and second lens frames 116 and 117, respectively. The frame 112 may also include a first lens frame 116 and a second lens frame 117 connected together by the nose bridge 115. The diagnostic eyewear 110 includes a first lens 121 which is non-magnifying, and a second lens 122 which is magnifying. The first and second lenses 121 and 122 may be held in place by the first and second lens frames 116 and 117. Alternatively, the first and second lens frames 116 and 117 may be fully or partially eliminated, e.g., the diagnostic eyewear 110 may be frameless or partially frameless.

FIG. 3 is a partially schematic isometric view illustrating diagnostic eyewear 210 in accordance with an embodiment of the present invention. Other than having generally circular lens frames, the features of the diagnostic eyewear 210 shown in FIG. 3 may be identical to the features of the diagnostic eyewear 10 shown in FIG. 1. The diagnostic eyewear 210 includes a frame 212 having a first temple piece 213, second temple piece 214, and nose bridge 215. The frame 212 may also include a first lens frame 216 and a second lens frame 217 connected together by the nose bridge 215. The first and second temple pieces 213 and 214 may be connected to the first and second lens frames 216 and 217, respectively. The diagnostic eyewear 210 includes a first lens 221 which is non-magnifying, and a second lens 222 which is magnifying. The first and second lenses 221 and 222 may be held in place by the first and second lens frames 216 and 217. Alternatively, the first and second lens frames 216 and 217 may be fully or partially eliminated, e.g., the diagnostic eyewear 210 may be frameless or partially frameless.

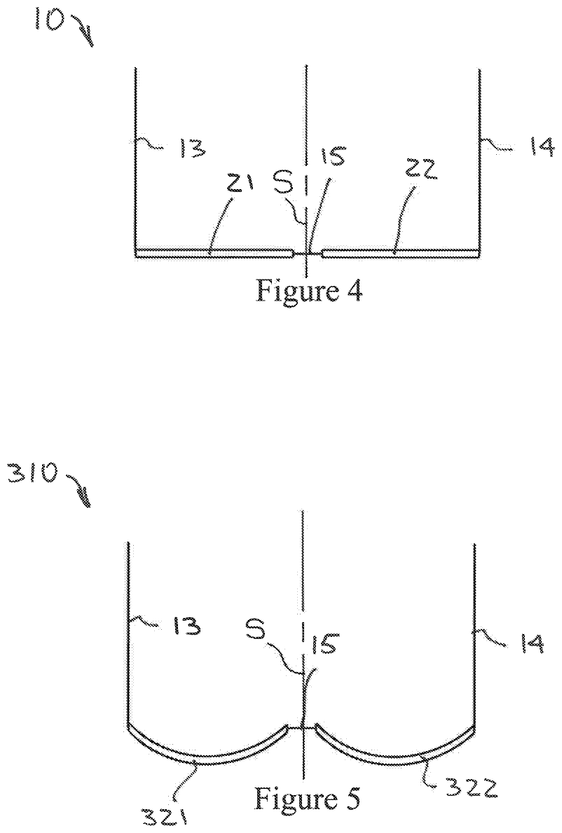

FIG. 4 is a partially schematic top view of the diagnostic eyewear 10, illustrating that the first and second lenses 21 and 22 are planar. FIG. 5 is a partially schematic top view of another embodiment of another embodiment of diagnostic eyewear 310, in which the first and second lenses 321 and 322 are curved. Thus, the shape of the lenses may be curved in the front as shown in FIG. 5, or flat in the front as shown in FIG. 4. For cosmetic purposes, it may be preferred to have lenses that are curved in the front. However, where the second position is obtained by rotating the temples, it may be preferred to have flat lenses.

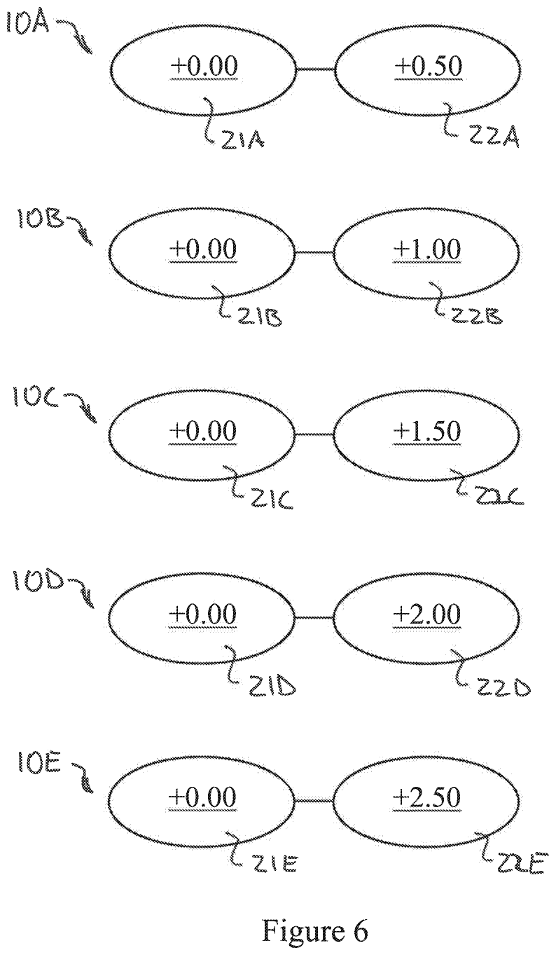

FIG. 6 schematically illustrates front views of a set of eyewear 10A, 1.0B, 10C, 10D, and 10E, in which the first lens is not magnifying and the second lens of each separate eyewear has a different magnification power. In the embodiment as shown, the diagnostic eyewear 10A includes a non-magnifying first lens 21A and a magnifying second lens 22A having a magnification of +0.50. The diagnostic eyewear 10B includes a non-magnifying first lens 21B and a magnifying second lens 22B having a magnification of +1.00. The diagnostic eyewear 10C includes a non-magnifying first lens 21C and a magnifying second lens 22C having a magnification of +1.50. The diagnostic eyewear 10D includes a non-magnifying first lens 21D and a magnifying second lens 22D having a magnification of +2.00. The diagnostic eyewear 10E includes a non-magnifying first lens 21E and a magnifying second lens 22E having a magnification of +2.50. In the eyewear set, each first lens of each pair of diagnostic eyewear comprises a plano lens, and the second lens has a magnification power ranging in 0.25 diopter increments from +0.25 to +10.00 or higher. Although the set shown in FIG. 6 includes five pairs of eyewear at diopters of +0.50, +1.00, +1.50, +2.00 and +2.50, any other desired number may be used. The magnification power of the second lens may be marked on the temple or any other part of the frame.

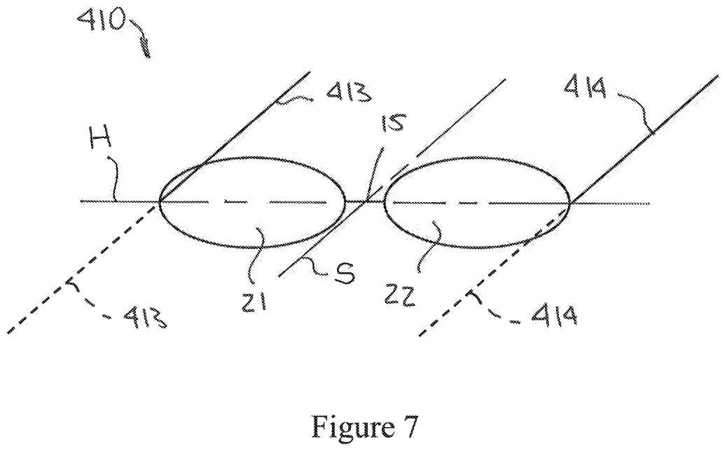

FIG. 7 is a partially schematic isometric view of diagnostic eyewear 410 in accordance with an embodiment of the present invention. In this embodiment, the diagnostic eyewear 410 includes a frame 412, reversible first and second temple pieces 413 and 414, and a nose bridge 415. As shown by solid lines in FIG. 7, the first and second reversible temple pieces 413 and 414 are in an initial rearwardly extending position. As shown in phantom in FIG. 7, the first and second reversible temple pieces 413 and 414 have been moved to forwardly extending positions 180° from the initial positions. The first and second temple pieces 413 and 414 may be pivoted from their rearward to forward positions in a horizontal plane P as shown in FIG. 7, in which case the temple pieces 413 and 414 pivot around vertical axes. Alternatively, the first and second temple pieces 413 and 414 may be moved in vertical planes from their rearward to forward positions, in which case the temple pieces 413 and 414 pivot around horizontal axes. However, any other suitable configuration may be used, such as pivoting the temple pieces 413 and 414 around non-vertical and non-horizontal axes, detaching and reattaching the temple pieces 413 and 414, or the like.

When the second position is obtained by moving the temples pieces 413 and 414 between rearward and forward positions as shown in FIG. 7, the nose bridge 415 does not need to be centered, nor does there need to be symmetry about the horizontal axis H. Instead, the nose bridge 415 may be arranged to allow the user to wear the diagnostic eyewear 410 with comfort in both the first and second positions.

The present invention provides several advantages. An embodiment of the present invention provides a method of determining magnification eye preference. In an embodiment, a person seeking monovision correction may be given the diagnostic eyewear. The person could have contact lenses correcting for nearsightedness. After receiving the diagnostic eyewear, the person may wear the diagnostic eyewear in the first position for a period amount of time, e.g., from several minutes to several hours or days, and then the person may wear the diagnostic eyewear in the second position for a period amount of time, e.g., from several minutes to several hours or days. The person could alternate the diagnostic eyewear from the first position to the second position until the person decides which eye is the preferred eye for magnification.

A further embodiment of the present invention provides a method of determining magnification strength preference. The person may be given a set of diagnostic eyewear, such as from three to five pairs of diagnostic eyewear, having different magnification strengths in the second lens of each pair of eyeglasses. The person may wear each pair of diagnostic eyewear until the person decides the magnification strength that is preferred.

In another embodiment of the present invention, a method of determining magnification eye preference and magnification strength preference can be performed in the same steps. For example, a person may be provided a set of the diagnostic eyewear wherein the second lens of each pair has a different strength than the other pairs. The person may wear one or more pairs of the diagnostic eyewear, as previously described, to determine both the magnification eye preference and the magnification strength preference.

For purposes of the detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about,” even if the term does not expressly appear. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

As used herein, “including,” “containing” and like terms are understood in the context of this application to be synonymous with “comprising” and are therefore open-ended and do not exclude the presence of additional undescribed or unrecited elements, materials, ingredients or method steps. As used herein, “consisting of” is understood in the context of this application to exclude the presence of any unspecified element, ingredient or method step. As used herein, “consisting essentially of” is understood in the context of this application to include the specified elements, materials, ingredients or method steps “and those that do not materially affect the basic and novel characteristic(s)” of what is being described.

Whereas specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the claims appended and any and all equivalents thereof.

What is claimed is:

1. Diagnostic eyewear for determining magnification eye preference for a wearer of the diagnostic eyewear to facilitate acclimation of the wearer to monovision, the diagnostic eyewear comprising:
a frame;
a first non-magnifying portion of the frame; and
a second magnifying lens supported by the frame,
wherein the frame is structured and arranged to alternatively position the first non-magnifying portion in a left viewing position in front of a left eye of the wearer while the second magnifying lens is positioned in a right viewing position in front of a right eye of the wearer, and then to position the first non-magnifying portion in the right viewing position while the second magnifying lens is positioned in the left viewing position.

2. The diagnostic eyewear of claim 1, wherein the first non-magnifying portion of the frame comprises a non-magnifying lens supported by the frame.

3. The diagnostic eyewear of claim 2, wherein the frame is 180° rotationally symmetric about a line of sight axis of the eyewear.

4. The diagnostic eyewear of claim 3, wherein the frame is 180° rotationally symmetric about a horizontal bisection line that intersects the line of sight axis.

5. The diagnostic eyewear of claim 2, wherein the frame comprises first and second temple pieces movable from first positions extending rearwardly from the first and second lenses to second positions extending forwardly from the first and second lenses.

6. The diagnostic eyewear of claim 5, wherein the first and second temple pieces are movable in horizontal planes from the first rearwardly extending positions to the second forwardly extending positions.

7. The diagnostic eyewear of claim 2, wherein the first non-magnifying lens is plano.

8. The diagnostic eyewear of claim 2, wherein the first non-magnifying lens is corrective.

9. The diagnostic eyewear of claim 8, wherein the first non-magnifying lens is corrective for nearsightedness.

10. The diagnostic eyewear of claim 2, wherein the second magnifying lens has a magnification power of from +0.25 to +10.00.

11. The diagnostic eyewear of claim 2, wherein the diagnostic eyewear is structured and arranged to determine distance eye preference of the wearer.

12. A plurality of diagnostic eyewear as recited in claim 2, wherein the second magnifying lens of each of the diagnostic eyewear has a different magnification power.

13. A diagnostic eyewear kit comprising a plurality of diagnostic eyewear, wherein each of the diagnostic eyewear comprises:
a frame;
a first non-magnifying portion of the frame; and
a second magnifying lens supported by the frame,
wherein the frame is structured and arranged to alternatively position the first non-magnifying portion in a left viewing position in front of a left eye of the wearer while the second magnifying lens is positioned in a right viewing position in front of a right eye of the wearer, and then to position the first non-magnifying portion in the right viewing position while the second magnifying lens is positioned in the left viewing position, and
wherein the second magnifying lens of each of the diagnostic eyewear has a different magnification power than the other second magnifying lenses.

14. The diagnostic eyewear kit of claim 13, wherein the first non-magnifying portion of the frame comprises a non-magnifying lens supported by the frame.

15. The diagnostic eyewear kit of claim 14, wherein the frame is 180° rotationally symmetric about a line of sight axis of the eyewear.

16. The diagnostic eyewear kit of claim 15, wherein the frame is 180° rotationally symmetric about a horizontal bisection line that intersects the line of sight axis.

17. The diagnostic eyewear kit of claim 14, wherein the frame comprises first and second temple pieces movable from first positions extending rearwardly from the first and second lenses to second positions extending forwardly from the first and second lenses.

18. The diagnostic eyewear kit of claim 17, wherein the first and second temple pieces are movable in horizontal planes from the first rearwardly extending positions to the second forwardly extending positions.

19. The diagnostic eyewear kit of claim 14, wherein the first non-magnifying lens is plano.

20. The diagnostic eyewear kit of claim 14, comprising from 2 to 10 of the diagnostic eyewear.

21. The diagnostic eyewear kit of claim 14, comprising from 3 to 6 of the diagnostic eyewear.

22. The diagnostic eyewear kit of claim 14, wherein the different magnification powers of the second magnifying lenses range from +0.25 to +10.00.

23. The diagnostic eyewear kit of claim 14, wherein the different magnification powers of the second magnifying lenses range from +0.50 to +5.00.

24. The diagnostic eyewear kit of claim 14, wherein the magnification powers of the second magnifying lenses have incremental differences of at least +0.10.

25. The diagnostic eyewear kit of claim 14, wherein the magnification powers of the second magnifying lenses have incremental differences of at least +0.25.

26. A method of determining magnification eye preference to facilitate acclimation to monovision, the method comprising:
placing the diagnostic eyewear of claim 1 on the wearer in a first one of the alternative positions; and
subsequently switching the position of the diagnostic eyewear to the other alternative position to thereby determine the magnification eye preference of the wearer.

27. The method of claim 26, further comprising determining distance eye preference of the wearer based upon the alternative placement of the diagnostic eyewear.

28. A method of determining desired magnification strength to facilitate acclimation to monovision, the method comprising:
placing a first diagnostic eyewear of claim 13 on the wearer in which the second magnifying lens has a first magnification power; and
placing a second diagnostic eyewear of claim 13 on the wearer in which the second magnifying lens has a second magnification power different from the first magnification power to thereby determine the desired magnification strength for the wearer.

29. The method of claim 28, further comprising determining magnification eye preference of the wearer by:
placing the first diagnostic eyewear of claim 13 on the wearer in a first one of the alternative positions; and
subsequently switching the position of the first diagnostic eyewear to the other alternative position to thereby determine the magnification eye preference of the wearer.

30. The method of claim 29, further comprising determining distance eye preference of the wearer based upon the alternative placement of the diagnostic eyewear.
